(19) United States
(12) Patent Application Publication
(10) Pub. No.: US 2010/0093873 A1
(43) Pub. Date: Apr. 15, 2010

(54) Methods of improving therapy of perfluorocarbons (PFC)

(76) Inventor: Sidney L. Goldfischer, New York, NY (US)

Correspondence Address: AMSTER, ROTHSTEIN & EBENSTEIN LLP, 90 PARK AVENUE, NEW YORK, NY, 10016, US

(21) Appl. No.: 12/587,034

(22) Filed: Oct. 1, 2009

Related U.S. Application Data
(60) Provisional application No. 61/194,955, filed on Oct. 2, 2008.

(51) Int. Cl. A61K 31/02; A61P 7/00

(52) U.S. Cl. 514/759

(57) ABSTRACT

This invention describes a novel, two-step method for administering PFC. The first step is designed to block the RES by administration of empty, small, liposomal vesicles (ESV) that are rapidly and preferentially engulfed by macrophages, thereby inhibiting their phagocytosis of subsequently infused PFC emulsions. The second step is the subsequent injection of PFC. ESV are devoid of materials that interfere with the macrophage's metabolic processes and do not impair their ability to clear the circulation of pathogenic organisms. Inhibition of the removal of PFC from the blood stream by the RES will achieve increased circulating PFC, enhanced binding and transport of oxygen throughout the blood stream and consequential reduction of undesirable consequences such as organomegaly and cytokine toxicity.

### CROSS-REFERENCE TO RELATED APPLICATION

This application claims the benefit of U.S. Provisional Patent Application No. 61/194,955, filed Oct. 2, 2008, the content of which is incorporated by reference.

### FIELD OF THE INVENTION

This invention relates to a method of increasing the half-life and concentration in the circulation of perfluorocarbon (PFC) oxygen-carrying emulsions, and the attenuation of certain side effects associated with the administration of PFC.

### BACKGROUND

The potential benefits of an artificial red cell substitute are numerous. For example, lost blood can be replaced quickly without the need for prior typing, without the risk of blood-typing errors, without fear of transmission of infected blood and without concern for the recurrent blood shortages. Particularly important for the treatment of trauma is the stability of modern PFC compounds that are instantly available, have a long shelf life and do not require refrigeration.

Perfluorocarbon compounds (PFC) have an extraordinary affinity for oxygen (Lane, T. A. 1995, Spahn, D. R. 1999). This property has stimulated extensive efforts to employ PFC as a red cell substitute and in other therapeutic roles in which the binding and transport of oxygen is critical (Winslow, R. M. 2006). In this applications, PFC is typically incorporated within phospholipid emulsions ranging in size from 0.10 microns to 0.25 microns; elimination half-times range from three to eight days (Spahn, D. R. 1999). Therapeutic attempts have met with limited success, in large measure because of side effects associated with the administration of the large amounts of PFC that have been given to overcome the short dwelling time of the emulsions in the circulation. This phenomenon is a consequence of the rapid uptake and prolonged storage of PFC by macrophages of the RES.

Accordingly, there exists a long felt need to improve the oxygen affinity of PFC by increasing its circulation half life, and to reduce side effects associated with PFC, so that PFC can be a viable red cell substitute.

### SUMMARY OF THE INVENTION

This invention is directed to improving the therapeutic oxygen transport and delivery efficacy of perfluorocarbon emulsions (PFC) in acute situations by increasing their dwelling time within the circulation. This is accomplished by the prior administration of empty liposomes that are rapidly taken up by the RES. Such retention inhibits the uptake by macrophages of subsequently administered PFC, thereby achieving therapeutic concentrations of the circulating oxygen carrier with lesser amounts of PFC and reduction of side effects associated with PFC. This invention is further directed to achieving the above while allowing macrophages to clear the circulation of circulating pathogens.

In this regard, the present invention is directed to a method for enhancing the oxygen transport capability of perfluorocarbon (PFC) artificial oxygen-carrying emulsions in a subject, by administering to the subject empty, small vesicles (ESV) in an amount and manner effective enhance the oxygen transport capability of the PFC. The oxygen transport capability may be increased by increasing the half-life (dwell-time) of PFC in the vascular system and/or by increasing the freely circulating concentrations of PFC. In the preferred embodiment, the ESV are sequestered by macrophages of the reticular endothelial system (RES), and more preferably, the sequestration of ESV by macrophages inhibits the capacity of the RES to phagocytose PFC. In accordance with the present invention, ESV is administered prior to PFC. Preferably, ESV and PFC are administered intravascularly.

The present invention is also directed to a method for increasing the half-life (dwell-time) of perfluorocarbon (PFC) artificial oxygen-carrying emulsions in the vascular system of a subject, by administering to the subject empty, small vesicles (ESV) in an amount and manner effective to increase the half-life (dwell-time) of PFC in the vascular system. In the preferred embodiment, the ESV are sequestered by macrophages of the reticular endothelial system (RES), and more preferably, the sequestration of ESV by macrophages inhibits the capacity of the RES to phagocytose PFC. In accordance with the present invention, ESV is administered prior to PFC. Preferably, ESV and PFC are administered intravascularly.

The present invention is further directed to a method for increasing freely circulating concentrations of perfluorocarbon (PFC) artificial oxygen-carrying emulsions in the vascular system in a subject, by administering to the subject empty, small vesicles (ESV) in an amount and manner effective to increase freely circulating concentrations of PFC in the vascular system. Again, in the preferred embodiment, the ESV are sequestered by macrophages of the reticular endothelial system (RES), and more preferably, the sequestration of ESV by macrophages inhibits the capacity of the RES to phagocytose PFC. In accordance with the present invention, ESV is administered prior to PFC. Preferably, ESV and PFC are administered intravascularly.

In addition, the present invention is directed to a method for reducing side effects associated with the administration of perfluorocarbon (PFC) artificial oxygen-carrying emulsions in a subject, by administering to the subject empty, small vesicles (ESV) in an amount and manner effective to reduce side effects associated with the administration of PFC. Such side effects include fever, flu-like symptoms and other adverse phenomena associated with the administration of PFC and enhanced release by RES macrophages of interleukin-1, tumor necrosis factor and other cytokines. In the preferred embodiment, the ESV are sequestered by macrophages of the reticular endothelial system (RES), and more preferably, the sequestration of ESV by macrophages inhibits the capacity of the RES to phagocytose PFC. In accordance with the present invention, ESV is administered prior to PFC. Preferably, ESV and PFC are administered intravascularly.

Still further, the present invention is directed to a method for attenuating hepatomegaly and/or splenomegaly that may occur following the administration of perfluorocarbon (PFC) artificial oxygen-carrying emulsions in a subject, by administering to the subject empty, small vesicles (ESV) in an amount and manner effective to attenuate hepatomegaly and/or splenomegaly that may occur following the administration of PFC. In the preferred embodiment, the ESV are sequestered by macrophages of the reticular endothelial system (RES), and more preferably, the sequestration of ESV by macrophages inhibits the capacity of the RES to phagocytose PFC. In accordance with the present invention, ESV is administered prior to PFC. Preferably, ESV and PFC are administered intravascularly.

The present invention is also directed to a method for treating a subject with perfluorocarbon (PFC) artificial oxygen-carrying emulsions, said method comprising administering to the subject empty, small vesicles (ESV), and subsequently administering to the subject perfluorocarbon (PFC) artificial oxygen-carrying emulsions. Preferably, the subject is need of treatment with a red cell substitute. In the preferred embodiment, the ESV are sequestered by macrophages of the reticular endothelial system (RES), and more preferably, the sequestration of ESV by macrophages inhibits the capacity of the RES to phagocytose PFC. In accordance with the present invention, ESV is administered prior to PFC. Preferably, ESV and PFC are administered intravascularly.

The present invention is also directed to a kit for therapeutic administration comprising (i) empty, small vesicles (ESV), and (ii) perfluorocarbon (PFC) artificial oxygen-carrying emulsions. 20. The kit may further include instructions for administration of the ESV and PFC, and preferably instructions to administer ESV prior to PFC.

### DETAILED DESCRIPTION OF THE INVENTION

According to the present invention, achieving effective inhibition of PFC uptake by macrophages will make it possible to achieve higher therapeutic concentrations of circulating PFC while administering smaller doses of PFC. Blockade of the macrophages of the RES is a well-known experimental phenomenon, but has not been employed as a therapeutic modality, largely because the RES functions as a primary defense against circulating pathogens. Nevertheless, materials, mainly lipids, exist which are preferentially taken up and stored within the RES without ill-effect. To be a therapeutically effective component of a PFC dosage regimen blockage of the RES uptake of PFC must not impede the ability of the RES to remove circulating pathogenic organisms from the blood stream. Blocking materials that are toxic to macrophages or significantly inhibit their ability to phagocytose pathogens are therefore not suitable for use in the present invention (Van Rooijenet al, 1990). The empty, small vesicles (ESV) for use in the present invention are preferably liposomes that are synthetic, multilamellar and/or unilamellar lipid microspheres that are preferentially engulfed by macrophages. For example, the synthesis of liposomes that vary in the extent to which they are taken up the RES by varying size, dosage, and lipid composition have been described (Wessef et al, 1984; Liu et al, 1992; Brand et al, 1194, which are hereby incorporated by reference in their entirety).

The present invention utilizes the initial enhancement, rather than suppression of phagocytosis of liposomes by the RES, thereby blocking the uptake by macrophages of PFC that is subsequently administered. It is the object of this invention to increase the half-life of circulating PFC incorporated within a lipid emulsion by inhibiting the uptake of such PFC by macrophages without significant injury to the macrophages. This can be accomplished by the administration of empty, negatively charged liposomes or liposomes with modified lipid bilayers that are preferentially taken up by macrophages (Proffitt et al, 1983, Van Etten, 1998) prior to the administration of PFC.

Macrophages engulf empty, negatively charged liposomes such as those made with the classic formulation of phosphatidylcholine, phosphatidylserine and cholesterol in a molar ratio of 40:10:50 more quickly and effectively than neutral lipid vesicles. For example, when a dose of such empty liposomes is administered to mice at a concentration of 80 micromole of lipid/kg, 90% of the liposomes are taken up by the RES in 30 min, a level of clearance that required 40 to 48 hours for other liposome formulations. Moreover, clearance of bacteria from the blood stream, a key function of the RES, is not impaired, even with a dose of negatively charged liposomes as high as 400 micromols/kg. K. pneumonaie and S. aureus were cleared from the blood stream as effectively as in control animals that did not receive any liposomes (van Etten et al 1998).

Effective, rapid blockade of the RES is also achieved by the use of empty, small, 0.1 micrometer, unilamellar liposomes that have 6-aminomannose incorporated in their lipid envelope. These vesicles are composed of distearoylphosphatidylcholine (DSPC), cholestorol (CH) and 6-aminomannose (8:3:1). When neutral lipid vesicles (DSPC:CH:diacetyl phosphate, 4:1:1) are subsequently injected they escape phagocytosis by the RES. (Proffitt et al, 1983).

The cited studies demonstrate that it is possible to block the RES with the administration of empty liposomes (SEV) without diminishing the RES capacity to clear circulating pathogens and permit subsequently injected PFC to largely escape capture by the RES, thereby enhancing the circulating concentrations of the oxygen carrier in the bloodstream.

This invention, which utilizes the prior administration of empty liposomes as a first step of a two-step process in the therapeutic administration of PFC in order to inhibit the uptake of PFC by the RES contrasts sharply with the disclosure of U.S. Pat. Nos. 5,679,394 and 5,624,220, both entitled, “Method of extending the vascular dwell time of particulate therapeutic and particulate diagnostic agents” (Long Jr., D. M. & Long, R. A. 1993, 1997). In these patents, liposomes infused with metabolically active particulates are administered simultaneously with the PFC emulsion in a one step procedure. By design their contents that (1) are deleterious to macrophages, (2) render the recipients susceptible to systemic infections, and (3) are co-administered together with the PFC. By contrast, in the methods of the present invention, (1) the blocking liposomes (ESV) are given prior to the infusion of PFC to inhibit the subsequent uptake of PFC and (2) the ESV are empty and devoid of materials that interfere with the metabolic activities of macrophages, to permit the RES to carry out its primary function as scavenger of circulating pathogens.

As discussed above, the present invention relates to a method of increasing the half-life and/or concentration in the circulation of perfluorocarbon (PFC) oxygen-carrying emulsions. The perfluorocarbon (PFC) oxygen-carrying emulsions that can be used in the present invention include, for example, the perfluorocarbon (PFC) oxygen-carrying emulsions disclosed in U.S. Pat. Nos. 4,859,363, 5,536,753, 5,621,144, 5,635,539, 5,628,930 and 7,383,395, which are hereby incorporated by reference in their entirety.

Therapeutic applications of such PFCs include, but are not limited to: (a) red cell replacement following trauma occurring on the battlefield or in accidents or in the course of surgery, (b) delivery of oxygen to ischemic tissues damaged by stroke and infarction (c) scavenging of oxygen bubbles in the circulation such as those generated during cardiac by-pass surgery that are responsible for mini-strokes with deleterious physical and psychiatric consequences (Newman et al 2001) and (d) delivery of high concentrations of oxygen to enhance the sensitivity of tumors to be subjected to radiation therapy. These therapeutic applications that require freely circulating PFC are limited by its short dwell time in the vascular tree. Metabolically inert PFC is readily engulfed and stored by RES macrophages necessitating the administration of excessive amounts of the oxygen carrier. The prolonged storage causes hepato- and splenomegaly and stimulates the release of tumor necrosis factor and other cytokines that are responsible for the deleterous side effects of PFC treatment.

Liposomes useful in the present invention are preferably small (generally under ten microns in diameter) multilamellar or unilamellar phospholipid vesicles and are preferentially engulfed by macrophages as described above. Empty, small vesicles (ESV) for use in the present invention can be prepared by standard techniques including, for example, as described in U.S. Pat. Nos. 4,814,270, 6,066,331 and 6,156,337, which are hereby incorporated by reference in their entirety.

The affinity of ESV for macrophages is the basis for the methods of the present invention. The first step is the administration of empty, small vesicles (ESV) that are devoid of particulates, antibiotics, metabolites or other materials that are toxic to the RES, but are selectively taken up by macrophages of the RES before treatment with PFC. The presence of phagocytosed ESV within macrophages inhibits their capacity to engulf subsequently administered PFC emulsions (Profitt, R. T. 1983; Lutz, J. 1994; NanRooijen, N. 1990). PFC will then remain within the circulation enabled to perform its intended function, the systemic transport and delivery of oxygen. The RES retains its ability to degrade the ESV and to clear the circulation of pathogenic organisms such as Klepsiella pneumococcus or staphyococcus aureus (Van Etten, 1998).

In another embodiment, the present invention is directed to a method to enhance the oxygen transport capability of intravascularly injected perfluorocarbon (PFC) artificial oxygen-carrying emulsions by increasing the half-life (dwell-time) of PFC in the vascular system. This method is accomplished by the prior intravascular administration of empty, small vesicles (ESV) that are sequestered by the macrophages of the reticular endothelial system (RES), thereby inhibiting the capacity of the RES to phagocytose subsequently injected emulsions of PFC and increasing the freely circulating concentrations of PFC and its therapeutic efficacy.

In yet another embodiment, the present invention is directed to a method to diminish the fever, flu-like symptoms and other adverse phenomena that occur following the administration of PFC emulsions and their phagocytosis by the RES. These symptoms occur as a consequence of the enhanced release by RES macrophages of interleukin-1, tumor necrosis factor and other cytokines. Because PFC is inert and cannot be metabolized both its storage within macrophages and their consequent release of cytokines is unduly prolonged. This method is accomplished by the intravascular administration of PFC only after the prior injection of ESV. The ESV are readily engulfed by the RES and the phagocytosis of PFC is blocked. As the ESV are readily broken down the stimulus for cytokine release is minimized.

In yet another embodiment, the present invention is directed to a method to diminish the hepatomegaly and splenomegaly that may occur following the administration of PFC. This method is accomplished by the intravascular administration of ESV prior to the injection of PFC, thereby inhibiting the uptake and retention of PFC by macrophages of the RES. ESV are non-toxic and unlike PFCs will be readily degraded by macrophage lysosomes.

The ESV for use in the present invention can be of varying sizes up to fifty or more microns in diameter and bounded by a unilamellar or a multilamellar membrane. The membrane may incorporate an aminomannose derivative of cholesterol, cholesterol, phosphtidylcholine or related lipids. The ESV are also devoid of any metabolite, antibiotic, enzyme or any other biologically active material. Again, the preparation of the ESVs can be prepared by conventional techniques. The quantities of ESV for use in the present invention will vary depending upon size and nature of the site targeted for PFC and the condition to be treated. A small, defined site such as the heart, brain, tumor or ischemic focus will require less PFC and ESV than what will be required to treat massive hemorrhage and battlefield trauma.

In summary, the life-saving potential for an artificial, oxygen-carrying substitute for red blood cells has resulted in the development of numerous perfluorocarbon (PFC) emulsions with an extraordinary capacity to bind and transport oxygen. Unfortunately, the clinical usefulness of PFC has been limited to a great extent by its short half-life in the circulation, mainly a consequence of the rapid clearance and prolonged storage of PFC by macrophages of the reticulo-endothelial system (RES). This invention relates to an improved method of delivering PFC to human subjects, thereby increasing its therapeutic efficacy, reducing the required amount of PFC and diminishing the adverse effects induced by PFC. PFC is inert and not metabolized, consequently storage within macrophages is greatly prolonged and causes hepatomegaly, splenomegaly and the sustained release of tumor necrosis factor and other cytokines.

### LIST OF CITED REFERENCES

Brandt, M. et al. 1994; Depression of the phagocytic activity of the RES by liposomes. Pharm Pharmacol Letter. 4: 1-4.

Lane, T. A. 1995; Perfluorochemical-based artificial oxygen carrying red cell substitutes. Transfus. Sci. 16: 19-31.

Liu, D. et al. 1992; Role of liposome size and RES blockade in controlling biodistribution and tumor uptake of GM1-containing liposomes. Bioch Biophys Acta. 1104: 95-101.

Lutz, J. et al. 1995; Acute toxicity and depression of phagocytosis in vivo by liposomes: influence of lysophosphatidylcholine. Life Sciences. 56: 99-106.

Newman, N. F., et al. 2002; Longitudinal assessment of neurocognitive function after coronary-artery bypass surgery. New Eng J of Med. 344: 395-402.

Proffitt, R. T. et al. 1983: Liposomal blockade of the reticuloendothelial system: Improved tumor imaging with small unilamellar vesicles Science. 220:502-505.

Spahn, D. R. 1999; Blood substitutes. Artificial oxygen carriers: perfluorocarbon emulsions. Crit Care 3: R93-R97.

Van Etten, E. M. W. et al. 1998; Administration of liposomal agents and blood clearing capacity of the mononuclear phagocytic system. Antimicrob Agents Chemother 42: 1677-81.

Van Rooijen, N. et al. 1990; Depletion and repopulation of macrophages in spleen and liver of rat after intravenous treatment with liposome-encapsulated dichloromethylene diphosphate. Cell Tissue Res. 260: 215-22.

Wassef, N. M. et al. 1984; Suppression of phagocytic function and phospholipids metabolism in macrophages by phosphatidylinositol liposomes. PNAS USA. 9:2665-2669.

Winslow, R. W. 2006; Current status of oxygen carriers (‘blood substitutes’) 2006. Vox Sanguinis 91: 102-110.

1. A method for enhancing the oxygen transport capability of perfluorocarbon (PFC) artificial oxygen-carrying emulsions in a subject, said method comprising administering to the subject empty, small vesicles (ESV) in an amount and manner effective enhance the oxygen transport capability of the PFC.

2. The method of claim 1, wherein the oxygen transport capability is increased by increasing the half-life (dwell-time) of PFC in the vascular system.

3. The method of claim 1, wherein the oxygen transport capability is increased by increasing the freely circulating concentrations of PFC.

4. The method of claim 1, wherein the ESV are sequestered by macrophages of the reticular endothelial system (RES).

5. The method of claim 4, wherein the sequestration of ESV by macrophages inhibits the capacity of the RES to phagocytose PFC.

6. The method of claim 1, wherein ESV is administered prior to PFC.

7. The method of claim 1, wherein ESV and PFC are administered intravascularly.

8-17. (canceled)

18. A method for reducing side effects associated with the administration of perfluorocarbon (PFC) artificial oxygen-carrying emulsions in a subject, said method comprising administering to the subject empty, small vesicles (ESV) in an amount and manner effective to reduce side effects associated with the administration of PFC.

19. The method of claim 18, wherein the side effects are selected from fever, flu-like symptoms and other adverse phenomena associated with the administration of PFC and enhanced release by RES macrophages of interleukin-1, tumor necrosis factor and other cytokines.

20-23. (canceled)

24. The method of claim 18, wherein the side effects are hepatomegaly and/or splenomegaly.

25-28. (canceled)

29. A method for treating a subject with perfluorocarbon (PFC) artificial oxygen-carrying emulsions, said method comprising administering to the subject empty, small vesicles (ESV), and subsequently administering to the subject perfluorocarbon (PFC) artificial oxygen-carrying emulsions.

30-32. (canceled)

33. A kit for therapeutic administration, said kit comprising (i) empty, small vesicles (ESV), and (ii) perfluorocarbon (PFC) artificial oxygen-carrying emulsions.

34. The kit of claim 33, which further comprising instructions for administration of the ESV prior to the PFC.
